(19) United States
(12) Patent Application Publication
(10) Pub. No.: US 2017/0246346 A1
(43) Pub. Date: Aug. 31, 2017

(54) A METHOD FOR PRODUCING A HAp (HYDROXYAPATITE)/BORON-DOPED HAp AND DEVELOPING COMPOSITE TISSUE SCAFFOLDS

(71) Applicants: Menemse GUMUSDERELIOGLU, Ankara (TR); Tugrul Tolga DEMIRTAS, Ankara (TR); Gokce KAYNAK, Ankara (TR); Ozge Ekin TUNCAY, Ankara (TR)

(72) Inventors: Menemse GUMUSDERELIOGLU, Ankara (TR); Tugrul Tolga DEMIRTAS, Ankara (TR); Gokce KAYNAK, Ankara (TR); Ozge Ekin TUNCAY, Ankara (TR)

(21) Appl. No.: 15/119,581

(22) PCT Filed: Feb. 24, 2015
(86) PCT No.: PCT/TR2015/000065

§ 371 Date: Aug. 17, 2016

(30) Foreign Application Priority Data
Feb. 24, 2014 (TR) 2014/02109

(51) Int. Cl. A61L 27/32; B05D 3/06; B05D 1/18; A61L 27/20

(52) U.S. Cl. CPC A61L 27/32; A61L 27/20; B05D 3/06; B05D 1/18; A61L 2430/02; A61L 2420/02

(57) ABSTRACT

The present invention relates to a method for producing biomimetic hydroxyapatite (HAp) and boron-doped HAp (B-HAp) with the support of microwave and a method for coating tissue scaffolds with HAp and/or B-HAp.

### TECHNICAL FIELD

The present invention relates to a method for producing biomimetic hydroxyapatite (HAp) and boron-doped HAp (B-HAp) with the support of microwave and a method for coating tissue scaffolds with HAp and/or B-HAp.

### BACKGROUND OF THE INVENTION

Hydroxyapatites (HAp) are used as bone filling material in a powdered state or in the form of porous block for filling cavities in bone structures in dental and orthopedic surgery or they are used as coating material for increasing binding with bond in metallic implant surfaces. When biological HAp structure is analyzed, it is detected that there are a large number of ions such as Na⁺, K⁺, Mg⁺, F⁻, Cl⁻ ve CO₃²⁻ in the structure. It is aimed to enhance osteoinductive characteristics of synthetic HAp, which is produced by addition of ions considered to support bone regeneration, into HAp structure. For synthesis of synthetic HAp, there are various methods such as sol gel approach, chemical coprecipitation method, hydrothermal method, multiple emulsion method, electrocrystallization method and biomimetic precipitation method.

Although there are a large number of studies concerning production of HAp in the literature, there is still no production method which correspond to HAp component in bone and studies continue on this subject. In studies included in the literature, HAp production is made using various salts with the help of microwave. However, no study has been done about HAp production by effect of microwaves on ions in SBF (simulated body fluid) and also no product has been developed using this method.

Boron regulates cell migration by affecting biological activation of cells and makes contribution to formation or regulation of ECM (extracellular matrix). Despite the fact that formation and production of biomaterials comprising boron in their composition came into question by making use of these above-stated effects of boron, studies done are in a limited number. Formation mechanism of apatite in SBF has been described by many researchers. It has been reported that materials, on the surfaces of which there are negatively charged functional groups (COO⁻, OH⁻), form nucleation site and support formation of apatite by enabling formation of amorphous calcium phosphate structure. However, the barrier for nucleation of homogeneous apatite from simulated body fluid is very high and a stimulation is required to induce heterogeneous nucleation. Therefore, additional chemical/physical processes are needed in most of the coating studies done using SBF. Despite all, it is not possible to coat particularly three dimensional (3D) porous tissue scaffolds with apatite homogeneously. Coating remains on the surface and it cannot penetrate to the depth of tissue scaffold throughout its pores. For this reason, it has not been possible to prepare composite tissue scaffold containing HAp effectively.

The Chinese patent document no. CN102557398, an application in the state of the art, discloses a medical support method and application thereof which comprises production of a boron-containing mesoporous and macroporous bioactive glass. The boron-containing porous bioactive glass is prepared by adding BO₃³— into SiO₄— by a sol-gel method. According to the method mentioned above, controllable porosity (78 to 90 percent) is obtained by adjusting immersion times (2-5 times) and pore sizes (300-500 microns). The mentioned glass filler material also enables to maintain structural integrity by providing nutrition transmission due to its porosity characteristic as well as it supports cell and tissue growth. When the boron content is changed from 0% to 10%, surface area of the boron-containing bioactive glass is changes from 365 to 194 m²/g. This is suitable for application of growth factors and drugs. The mentioned biological material enables to release of boron ions slowly and it can also be used as a tissue repair material in bone and periodontium engineering. In the mentioned patent document, the boron-containing bioactive glass with bioactive structure is prepared by a sol-gel method. It is anticipated that this bioactive structure, which is prepared by substitution of silicon oxides in glass with boron oxide, can be used as bone filler and tissue scaffold.

The Chinese patent document no. CN87107744A, another application in the state of the art, discloses a biological adaptability for composite material. This material comprises a glass layer on an upper hydroxyapatite ceramic layer substrate and an intermediate glass layer between the substrate and the layer. The glass-hydroxyapatite ceramic layer comprises a hydroxyapatite ceramic which is dispersed such that it will form a continuous glass phase. The mentioned hydroxyapatite ceramic material comprises Ca/P in a molar ratio of 1.5 to 1.75. Unused spaces and hydroxyapatite-containing parts of the layer are exposed to a roughened effect. The composite material production method containing hydroxyapatite-coated part of the substrate and powdery glass comprises obtaining the coated substrate by sintering and by acid etching. The composite materials can be used for treatment of bone defects for bone substitute material. The mentioned invention discloses developing composite materials in order to be used for coating metal-based orthopedic implants by sintering glasses having powdered different components with hydroxyapatite.

### SUMMARY OF THE INVENTION

An objective of the present invention is to realize a method which enables to obtain a bone-like hydroxyapatite (HAp) structure using microwave-supported biomimetic method from simulated body fluid differently from conventional methods so as to be used in bone tissue repair.

Another objective of the present invention is to realize a method which enables to obtain a boron-added hydroxyapatite (B-HAp) of boron mineral, that has many functions in human metabolism and particularly is considered to have osteoinductive characteristic, upon being incorporated in HAp structure by SBF (simulated body fluid) and microwave energy.

A further objective of the present invention is to realize a method whereby other ions (Fe, Sr, Mg, etc. . . . ) which are necessary for bone other than boron are also incorporated in HAp structure by SBF (simulated body fluid) and microwave energy.

A yet further objective of the present invention is to realize a method which enables to obtain structures of composite bone tissue scaffolds owing to the fact that 3D, porous tissue scaffolds (all polymeric materials) can be coated with HAp/B-HAp by making use of microwave support and without using an additional agent triggering nucleation, in a rapid and homogeneous way.

### DETAILED DESCRIPTION OF THE INVENTION

In order to obtain HAp for bone tissue repair by biomimetic method, the steps of:
preparing a SBF (simulated body fluid) solution which comprises a Ca/P (calcium/phosphate) concentration 10 times more than blood plasma:
adding NaHCO₃ into the solution before the precipitation process;
to exposure the solution to a microwave process and carrying out the precipitation process
   are followed.

In the inventive method, a 10×SBF-like solution comprising Ca/P concentration 10 times more than blood plasma is selected as solution due to negative effect of simulated body fluid's (SBF) saturation level on Ca/P nucleation and precipitation. There are components of NaCl, KCl, CaCl₂.2H₂O, MgCl₂.6H₂O and NaH₂PO₄.H₂O in the mentioned solution. Order of addition and amount (g) to be added and concentration (mM) values of the mentioned components are given in the Table 1. Firstly NaCl, secondly KCl, thirdly CaCl₂.2H₂O, then MgCl₂6H₂O and lastly NaH₂PO₄.H₂O are added when preparing the mentioned solution. First five salt components are dissolved in a magnetic stirrer within 800 mL distilled water respectively and it is completed to 1000 mL by addition of 200 mL distilled water in preparation of solution. In a preferred embodiment, the solution is prepared as a stock solution and it can be maintained within a storage box at +4° C. or room temperature for a long time.

TABLE 1
10xSBF-like solution prescription

| | Order of | Amount | Concentration |
|---|---|---|---|
| Components | Addition | (g) | (mM) |
| NaCl | 1 | 58.443 | 1000.00 |
| KCl | 2 | 0.373 | 5.00 |
| CaCl₂•2H₂O | 3 | 3.675 | 25.00 |
| MgCl₂•6H₂O | 4 | 1.016 | 5.00 |
| NaH₂PO₄•H₂O | 5 | 0.250 | 3.62 |
| NaHCO₃ | 6 | 0.084 | 10.00 |

In a preferred embodiment of the invention, 100 mL stock SBF solution is taken into a glass beaker and addition of NaHCO₃ is made such that its concentration will be 10 mM before the precipitation process.

The precipitation process is realized and HAp is obtained by inducing biomimetic process with microwave irradiation. Precipitation conditions are given in the Table 2. Precipitation is occured by applying the microwave processes to the solution by various powers, various times and number of repetition. To make comparison, HAp is also produced by applying conventional heating to the solution (holding at 80° C. for 1 hour and 2 hours) or performing precipitation for 6 hours at room temperature without applying microwave irradiation.

TABLE 2
Production conditions of microwave-supported HAp

| Power (W) | Time (s) | Number of repetition |
|---|---|---|
| 90 W | 300 | 6 |
| 360 W | 60 | 5 |
| 360 W | 60 | 10 |
| 600 W | 30 | 4 |
| 600 W | 30 | 7 |
| 600 W | 30 | 9 |
| 600 W | 30 | 12 |
| 600 W | 30 | 15 |
| 1200 W | 15 | 9 |

In order to remove other undesirable phases after the precipitation process, the solution is washed and allowed to dry. In a preferred embodiment, the solution is washed by being centrifuged at 5,000 rpm for 5 minutes within ethanol for 2 times and distilled water for 3 times and it is allowed to dry in drying-oven at 37° C. temperature for the purpose of removing other undesirable phases.

Salts are added in accordance with the order given in the Table 1 in order to prepare a 10×SBF -like solution after H₃BO₃ (boric acid) and NaOH (sodium hydroxide) are added into 1000 mL ultrapure water so as to obtain a bone-like B-HAp. In order that precipitation occurs, microwave process is applied to the solution. Addition of NaHCO₃ is made into the solution for the purpose of providing a suitable pH medium so that the precipitation process occurs. Besides the variables such as the microwave power, time and number of repetition applied in the Table 3, different H₃BO₃ additions and amounts of NaOH and NaHCO₃ necessary to be added in order to provide pH (6.5-7.4) for suitable precipitation are given. In order to remove other undesirable phases after the precipitation process, the solution is washed and allowed to dry and then B-HAp is obtained.

TABLE 3
Production conditions of microwave-supported B-HAp

| H₃BO₃ | NaOH | NaHCO₃ | Power | Time | Number of | SBF |
|---|---|---|---|---|---|---|
| (mg)* | (10M) (μL) | (g) | (W) | (s) | repetition | used |
| 5 | — | 0.0840* | 600 | 30 | 9 | 10xSBF |
| 10 | — | 0.0840* | 600 | 30 | 9 | 10xSBF |
| 10 | — | 0.0840* | 600 | 30 | 15 | 10xSBF |
| 10 | — | 0.0840* | 1200 | 15 | 9 | 10xSBF |
| 10 | — | 0.0840* | 1200 | 15 | 15 | 10xSBF |
| 10 | 900 | 0.0840* | 600 | 30 | 9 | 10xSBF |
| 17 | — | 0.0940* | — | — | — | 10xSBF |
| 17 | — | 0.1040* | 600 | 30 | 9 | 10xSBF |
| 17 | — | 0.0840* | — | — | — | 10xSBF |

In the invention, it is critical that pH of SBF is in the range of precipitation pH in HAp production by biomimetic method. Otherwise, the precipitation process does not occur. Amounts of H₃BO₃ (boric acid), NaHCO₃ (sodium bicarbonate) and NaOH should be added by paying attention not to break this balance.

The coating process is carried out by subjecting the chitosan tissue scaffolds, which are prepared by freeze drying method, to microwave process in SBF solution and type composite scaffolds are obtained in the inventive method for producing HAp/chitosan and/or B-HAp/chitosan coated composite tissue scaffolds with the help of microwave.

In the freeze drying process, firstly 2% (w/v) chitosan (75-85% degree of deacetylation) solution of chitosan tissue scaffolds are prepared using distilled water comprising 0.2 M acetic acid. The solution obtained is filtered so as to be made free from impurities and it is frozen at −20° C. after being poured into 24-well Petri dishes. After this step, chitosan tissue scaffolds are subjected to the process of “freeze drying” at −80° C. using lyophilizer (freeze dryer). Because the ice crystals inside the scaffolds are lyophilized in this process, a porous structure having intrinsic connections is obtained. The scaffolds are hold in 96% (v/v) ethanol solution for 1 day whereas in 70% (v/v) ethanol solution for 1 hour for stabilization of the chitosan tissue scaffolds obtained.

In a preferred embodiment, after they are cut by dies in 2 mm×9 mm sizes the tissue scaffolds are precipitated in a vacuum drying-oven within stock solutions wherein no NaHCO₃ is added, it is ensured that air inside thereof is taken out and all pores are contacted with SBF solutions. Afterwards, the scaffolds are transferred into 100 mL SBF and B-SBF solutions wherein NaHCO₃ is added, coating is carried out with the help of microwave such that there will be 7 scaffolds/100 mL solution (SBF and B-S13F). Coating with the help of microwave is realized under conditions wherein production of HAp closest to natural bone structure is made and highest boron contribution can be provided as a result of characterization of the HAp and B-Hap phases produced. Due to the fact that a HAp structure with a composition closest to bone is obtained under a condition where 600 W power is applied.

Carboxyl groups included in organic polymers such as chitosan stimulate apatite nucleations. Ca²⁺ is bounded over carboxyl groups whereas P0₄³⁻ groups are bounded over amine groups primarily. However, carboxyl groups also need to be stimulated. With the support of microwave irradiation used the scope of the present study, desired HAp and/or boron-doped HAp structure is obtained and also sufficient and effective coating is provided in chitosan tissue scaffold with macro pore.

With the inventive method, an effective coating process is carried out by use of microwave energy without needing a modification such as forming cylanol (Si—OH) groups on the surface of chitosan.

It is aimed to prevent formation of undesirable phases and obtain HAp and B-HAp strictures in a short time as a result of interaction of microwave energy with ions within SBF by means of the invention. It is not necessary to keep physiological pH during processes in hydroxyapatite production. A 10×SBF-like solution is preferred because of advantages such as: it is not necessary to use buffers such as Tris and HEPES which are not included in blood plasma, precipitation occurs fast depending on saturation level, and CO₂ supply is not required. By means of energy supplied from outside by microwave, HAp formation mechanism occurring by diffusion normally is carried out faster by accelerating atomic motion and reducing diffusion barrier. Thus, it is ensured that intermediate steps are skipped in HAp formation mechanism and a biocompatible “bone-like hydroxyapatite” which imitates bone structure and can be obtained in an easy, fast and effective way in high amounts is obtained instead of a HAp structure which is obtained in a long time by chemical methods.

In order to determine advantages or disadvantages of microwave method, analysis of HAp structures produced by other methods are were carried out and the results obtained were compared. When ICP-OES analysis was applied to the examples which had been produced as a result of trials carried out under different conditions (90 W (5 min×6), 360 W (1 min×5), 600 W (30 h×9), 1200 W (15 h×9), conventional method (1 h 80° C.) and 6 h precipitation at room temperature); a value closest to bone was obtained in the group of 600 W (30 h×9).

Besides, boron which has an osteoinductive characteristic plays role in bone metabolism is doped into hydroxyapatite structure. Thus for, boron-doped hydroxyapatites have been synthesized via chemical methods by man), researchers but there is no study whereby interaction thereof with cells. In our studies done, an inefficient precipitation was observed in 37° C. at 24 hours in boron added 10×SBF solution and no precipitation was observed when it was kept at room temperature whereas an effective and rapid precipitation was determined with the support of microwave. For the purpose of making sufficient boron addition, addition of boric acid is made to SBF in amounts that will not to affect ion balance (10 mg/mL) and after pH values (6.5-7.4) necessary for precipitation were provided, it was ensured that hydroxyapatite with desired structure precipitated upon being subjected to microwave. In the studies which were done for the purpose of making pH of the boric acid-added simulated body fluid suitable for precipitation; 10 mg/mL boric acid was added before salts were added into ultrapure water, pH was measured at this stage and average value was detected as 5.90. pH was detected as 7.59 on average and pH was detected as 6.8 after neutralization with 900 μL NaOH (10M). pH was increased around 7.01 by addition of 0.0840 g NaHCO₃ added before precipitation and precipitation was realized with microwave effect.

In this part of the detailed description, results obtained following experimental studies on characterization of the inventive bone-like HAp and B-HAp and values in relation to these results will be presented by means of tables and figures.

Ratios of Ca/P are important factors in biomaterials to be used as bone substitution and the mentioned ratios give information about the phase impurity, the chemical homogeneity and solubility of the material. Human bone comprises 34.8% Ca, 15.2% P ion by weight (w/w) and the molar ratio of Ca/P is approximately around 1.77; stoichiometric hydroxyapatite comprises 39.8 Ca%, 18.5% P ion and the ratio of Ca/P is approximately 1.66. Biologic hydroxyapatites show deviation from stoichiometric structure by getting into substitution reactions with the carbonate groups or other cationic or ionic groups incorporated into their structures.

Ratios of % Ca, % P and Ca/P obtained from the ICP-OES (inductively coupled plasma optical emission spectroscopy)analysis of the inventive HAp is given in the Table 4. HAp produced with the support of microwave irradiation has Ca²⁺ deficient and bone-like values while examples produced with precipitation at conventional heating and room temperature have less Ca/P ratio. Particularly, examples produced at 600 W power have the value (1.61) closest to bone. In the study done, this ratio has been increased to 1.61 using microwave energy. In addition, ratio of % Ca and % P in human bone varies by type of bone. In the studies, it was reported that cortical bone has 23.0±3.9% wt. Ca and 10.7±2.4% wt. P weight contens while trabecular bone has approximately 11.4±2.8% wt. Ca and 5.58±1.49% wt. P. In consideration of this information, it can be concluded that HAp production with the support of microwave provides HAp formation closer to cortical bone.

TABLE 4
Ratios of %Ca, %P and Ca/P included
in stoichiometric hydroxyapatite.

| | Ca (%) | P (%) | Ca/P |
|---|---|---|---|
| Example | (w/w) | (w/w) | (Molar ratio) |
| Precipitation at room temperature | 30.1 ± 0.3 | 17.2 ± 0.3 | 1.35 |
| Conventional method | 31.0 ± 0.1 | 15.6 ± 0.1 | 1.53 |
| 90 W | 29.7 ± 0.5 | 15.9 ± 0.9 | 1.44 |
| 360 W | 29.6 ± 0.2 | 14.6 ± 0.4 | 1.56 |
| 600 W | 31.5 ± 0.4 | 15.1 ± 0.3 | 1.61 |
| 1200 W | 27.8 ± 0.4 | 14.0 ± 0.6 | 1.53 |
| Bone^{a} | 34.8 | 15.2 | 1.77 |
| HAp^{b} | 39.8 | 18.5 | 1.66 |

% Ca, % P, % B included in the structure of B-HAp, another subject of the invention, and ICP-OES analysis results of their ratios are shown in the chart Table 4.1.

TABLE 4.1
% Ca, %P, %B, Ca/P, Ca/P + B, P/B ratios included in B-Hap examples.

| | | | | Microwave conditions | | | | | | | |
|---|---|---|---|---|---|---|---|---|---|---|---|
| | | | | | Application | | | | | | |
| H₃BO₃ | NaOH | NaHCO₃ | | Power | repetition × | Ca(%) | P(%) | B(%) | | | |
| (mg)* | (10M) (μL)** | (g) | pH | (W) | time | (w/w) | (w/w) | (w/w) | Ca/P ^{#} | Ca/P + B^{#} | P/B^{#} |
| 5 | — | 0.0840* | 6.40 ± 0.03 | 600 | 9 × 30 s | 30.45 ± 0.64 | 14.90 ± 0.00 | 0.47 ± 0.13 | 1.58 | 1.45 | 11.61 |
| 10 | — | 0.0840* | 6.21 ± 0.02 | 600 | 9 × 30 s | 31.64 ± 2.90 | 16.65 ± 1.70 | 0.37 ± 0.16 | 1.47 | 1.38 | 19.79 |
| 10 | — | 0.0840* | — | 600 | 15 × 30 s | 32.68 ± 1.59 | 16.43 ± 1.37 | 0.39 ± 0.09 | 1.54 | 1.44 | 15.20 |
| 10 | — | 0.0840* | — | 1200 | 9 × 15 s | 30.80 ± 0.28 | 15.25 ± 0.07 | 0.22 ± 0.08 | 1.57 | 1.50 | 26.47 |
| 10 | — | 0.0840* | — | 1200 | 15 × 15 s | 31.10 ± 0.57 | 15.45 ± 0.21 | 0.25 ± 0.09 | 1.56 | 1.49 | 22.61 |
| 10 | 900 | 0.0840* | 7.02 ± 0.03 | 600 | 9 × 30 s | 28.10 ± 0.28 | 15.55 ± 0.35 | 1.15 ± 0.11 | 1.40 | 1.16 | 4.75 |
| 17 | — | 0.0940* | 6.01 ± 0.02 | — | — | — | — | — | — | — | — |
| 17 | — | 0.1040* | 6.05 ± 0.05 | 600 | 9 × 30 s | 25.80 ± 1.27 | 13.80 ± 0.14 | 0.181 ± 0.04 | 1.44 | 1.39 | 27.40 |
| 17 | — | 0.0840* | 5.92 ± 0.02 | — | — | — | — | — | — | — | — |

*For 100 mL SBF
**For 1 L SBF.
^{#}Ca/P, Ca/P + B and P/B ratios represent ratios by mole.

Morphologies of the inventive HAp and B-HAp examples were determined by scanning electron microscope. Images of the powdered samples in the FIG. 1 are reached at different magnifications.

SEM results indicated that the samples obtained by precipitation from SBF form clusters (0.5-4 μm) by gathering. No morphologically significant difference was detected in the structures of bulk HAp and B-HAp. HAp development continues over these nucleation sites after nano apatite crystals are formed in the solution, thus HAp nano particles create clusters in micron sizes.

It was observed that undesirable salt crystals in different sizes also occur in the coating, which was performed on glass coverslip so as to examine the structure better, these crystals were easily removed by washing processes. In a coating made by another SBF prescription (10×SBF, Tas and Bhaduri, 2004) it was determined that undesirable crystals occurring in the form of a one-piece layer cannot be removed in consequence of washings performed (Mavis et al., 2009). (FIG. 1.1).

In order to examine crystal structures of the inventive HAp and B-HAp examples better, “transmission electron microscope (TEM)” images were taken. Due to the fact that nano-sized particles occurred create hydroxyapatite clusters by combining, existence of nano-crystal structures not clear in SEM images was observed by TEM analysis clearly (FIG. 2).

Nano/microcrystal HAp structures having various morphologies such as nanowire, nanorod, microsphere, micro flower and micro layer have been synthesized so far by many different methods. Whereas HAp in bone structure has a rod-like nanocrystalline morphology. TEM images of our samples produced with the help of microwave irradiation showed that they were similar to biological apatite with their needle-like structure and upon the power applied in the microwave increases, length of nanocrystals shorten and they start taking the shape of rod instead of needle-like morphology. In the literature, crystal sizes of biologic apatite are stated as length of 30-50 nm, width of 15-30 nm and thickness of 2-10 nm. It is seen that samples, produced by application of 600 W, have length of 40-50 nm on average similar to the apatite structure in bone. In measurements made by Zeta sizer, average particle sizes of nanocrystals were measured bigger due to coming together. In analysis which were carried out by the samples prepared by dispersing the HAp directly inside distilled water, average particle size was measured as 611.5±21.35 nm; when sodium dodecyl sulphate (SDS) was used and dispensed by ultrasonic application combining clusters separated, particle sizes decreased and average size was measured as 183.2±33.89 nm.

No significant difference was observed between the morphologies of HAp and B-HAp produced under conditions of 600W 9×30 sn with the help of microwave when TEM images were compared. With boron addition, hydroxyapatite nanoparticles changed from needle-like morphology to rod-like morphology. Also, particle size of hydroxyapatite on average was on the length of 40-50 nm (FIG. 2a, b and c) whereas particle size of boron-added hydroxyapatite decreased to 20-30 nm (FIG. 2.1d, e and f).

XRD graphics of the inventive HAp and B-HAp samples obtained as a result of the X-ray diffraction (XRD) analysis are given in the FIG. 7.The peaks observed around 2θ=26° and 32° which are characteristic peaks belonging to hydroxyapatite.

All samples except the 360 W power was applied a single HAp structure was observed, no other structure was seen. In the sample wherein 360 W power was applied, a biphasic CaP structure comprising HAp and tricalcium phosphate (TCP) was observed. It was considered that effect of 1 minute application on reaction kinetic was not sufficient at this power.

High wide band indicated that the apatite phase has an amorphous structure instead of crystalline structure and this structure indicates high similarity with biological apatite. Increase of the power applied in microwave indicates (1200 W) increase of shapness of wide peaks and HAp phase crystallinity.

XRD graphics of HAp and B-HAp produced in selected conditions (600W) were comparatively given in the FIG. 3.1. When the spectrums were examined, it was determined that boron addition does not cause formation of different phases such as dicalcium phosphate anhydrous (DCP), dicalcium phosphate dihydrate (DCDP) or P-tricalcium phosphate ((3-TCP) and only single-phase (comprising only HAp) structures occur.

Stoichiometric hydroxyapatite has a totally crystalline structure however the hydroxyapatite and the boron-added hydroxyapatite crystal produced within the scope of the present study has no crystalline structure. When XRD patterns were examined, it was clearly seen that peaks were very sharp in the stoichiometric hydroxyapatite but it was not sharp HAp and B-HAp (FIG. 3.1.c). Decreases in peak expansion and peak sharpness showed similarities with natural bone structure. These bone-like apatites have semi-crystalline characteristics. It was determined that boron addition does not lead to any change in hydroxyapatite crystallinity.

Attenuated total reflection Fourier transform infrared spectroscopy (ATR-FTIR) analysis (ATR-FTIR) spectrums of the carbonated hydroxyapatites were give in the FIG. 4. All characteristic peaks of water, hydroxyl, phosphate and carbonate groupor related to HAp.

When ATR-FTIR spectrum is examined, the common peak was seen in the range of 3000-3600 cm⁻¹ which indicates existence of adsorbed water into HAp structure and the peak was observed at ˜1640 cm⁻¹ that indicates existence of unbound water related to HAp. Bound water weakens the bond between calcium and hydration ball around thereof by absorbing electromagnetic energy through microwave radiation and thus it is ensured that dehydration step necessary for apatite formation in aqueous solutions is realized. Effect of microwave energy on free water, which is generated by bound water bounded by hydrogen bonds in the form of hexagonal ring, is limited by heating and evaporation. Energy absorbed by free water increases as power of microwave increases. Therefore, time required by bound water to absorb energy depends on temperature increase occurring with microwave absorbance by free water.

In addition, the peaks observed around 954 and 1020 cm⁻¹ exhibits more narrow and sharp distribution with increase of power from 90 W to 1200 W indicates that crystallinity of hydroxyapatite increases such that it will support XRD results.

The adsorbtion bands at ˜560, 601, 954, 1020 cm⁻ detected in spectrums attributed to the phosphate (PO₄⁻³) ion in the structure and at around 1470-1410 cm⁻ and 870 cm⁻ suggest the precence of carbonate (CO₃⁻²) ion which is supposed to hane come SBF and atmosphere during precipitation process. When FIG. 3.1 is examined, it can be seen that carbonate peak in the HAp structure obtained by precipitation at room temperature is not apparent. In this case, carbonate addition into the structure becomes much lower than addition into the apatite obtained by microwave effect.

Biological apatite are usually carbonate-substituted Ca²⁺ deficient hydroxyapatite and significant amounts of carbonate (4-8%) are always present in the mineral part of bone. Also substitution of carbonate into HAp structure changes its crystallographic parameters and morphology, decreases crystallinity increases its solubility causes charge imbalance and it may also increase mechanical strength. The charge imbalance may be sourced by calcium vacancies. This is the reason why Ca²⁺ deficient HAp synthesize in the presence of SBF. Some monovalent ions such as Na⁺ substituted to Ca⁺² sites for compensating this vacancies. Incorporation of carbonate into structure was carried out with two different ways. These carbonated apatites have been identified type A or B HAp. While carbonate ions substitutes onto the OH⁻ sites in type A, they substitutes onto the PO₄⁻³ sites in type B. Ratio of A/B in the human bone may vary depending on the age factor.

If CO₃⁻² peak in FTIR spectrums is seen in 884, 1465, 1534 and 1550 cm⁻¹ values it indicates that it is A Type while it indicates that it is B Type if it is seen in 864, 1430, 1445 CO₃⁻² and 1534 cm⁻¹ values. In the structure produces as a consequence of interaction with microwave within the scope of the present study, it was observed that carbonate ion mostly substitutes OH⁻¹ and the band was observed at 872 cm⁻¹ value and also peaks exhibited some deviation according to the values provided in the literature. Existence of weak OH⁻ peak in the range of 3000-3600 cm⁻¹ also supports apatite structure of B Type.

In the FTIR spectrum given in the FIG. 5, asymmetric streching and symmetric bending bands of BO₃³⁻ groups at around 1304, 1253, 1208 cm⁻¹ and 784, 771, 755 cm⁻¹ are expected to be seen, respectively. Whereas bands expected to be seen in 2002 cm⁻ and 1932 cm⁻ are asymmetric streching bands of BO₂⁻ group. Although existence of BO₃³⁻ in B-HAp structure and BO₂⁻ incorporation gaps of crystal structure is shown by ICP-OES analysis, borate peaks were not observed in FTIR spectrum due to the fact that both it is included in the structure about 1.15% and peaks of borates are very weak. However, it is clearly seen that carbonate peaks are more obvious in HAp spectrum when HAp spectrum and B-HAp spectrum are compared. Thus, substitution of BO₃³⁻ ion with PO₄³⁻ or OH⁻ competitively—which is also mentioned in the results of elemental analysis—and accordingly decrease of CO₃²⁻ amount in the structure of B-HAp are also supported by the results of ATR-FTIR.

Because there is carbonate ion in the biological apatite structure in the invention, it is important to incorporate carbonate into the stricture for characterizing the hydroxyapatite as bone-like and various studies were done for this purpose. Results of the elemental analysis, which was carried out in order to determine the amount of carbonate included in the examples produced in the study, are given in the Table 5.

TABLE 5
%CO₃⁻² ratios by weight of the examples which
are produced within the scope of the present study

| Example | CO₃(%) (w/w) |
|---|---|
| Precipitation at room temperature | 5.15 ± 1.69 |
| Conventional method | 12.07 ± 0.67 |
| 90 W | 11.62 ± 0.60 |
| 360 W | 10.08 ± 1.78 |
| 600 W | 9.22 ± 0.47 |
| 1200 W | 9.77 ± 1.84 |

Compared to the carbonate content of 4-8% in natural bone structure, it was determined that structure of hydroxyapatite produced using particularly 600 W power by microwave shows similarity with bone structure in terms of carbonate content. It was ensured with use of microwave in production of HAp from simulated body fluid that incorporation carbonate into the structure increased double compared to precipitation at room temperature. The elemental analysis carried out yielded a result compatible with the ATR-FTIR analysis.

Carbonate contents of HAp and B-HAp, which are selected to be used also for cell culture studies in the study, are given in the Table 6.

TABLE 6
%CO₃²⁻ contentsof HAp and B-HAp samples

| Example | CO₃²⁻ (w/w, %) |
|---|---|
| HAp | 9.22 ± 0.47 |
| B-HAp | 4.30 ± 0.07 |

The reason of decrease of carbonate content in structure of boron-doped hydroxyapatite is substitution of CO₃²⁻ ions with BO₃³⁻ ions with our or PO₄³⁻ ions competitively. This result is also compatible with the carbonate peaks in the ATR-FTIR analysis.

Raman and infrared spectroscopy analysis carried out present studies complement each other. During interaction of molecules with a severe monochromatic light beam, if no light absorption occurs then light scattering takes place. During light scattering, energy of a major part of the light scattered is equal to the energy of the light interacting with the material and this type of scattering event is called as “Rayleigh scattering”. Besides the scattering event only a small part of the light scattered scatters with energies more different than the light interacting with molecule. This type of non-elastic scattering event is called as “Raman scattering”. In the energy of the light scattered during Raman scattering, excess or shortage taking place according to the light scattering with molecule is as much as the energy difference between the vibrational energy levels of the molecule scattering with the light. Therefore, information can be obtained about vibrational energy levels of the molecules by spectroscopic examination of “Kaman scattering”.

In order that molecules can absorb infrared photon, in other words the molecule can get into resonance with this photon, there must be a change which is periodical in the dipole moment and has a frequency equal to the frequency of the photon while the molecule s vibrating. Whereas in order that a molecule can get into a Raman type of scattering interaction with a photon, the photon with which the molecule interacts during vibration thereof needs to be polarized periodically and with a frequency equal to the frequency of this photon by electrical field in other words a periodical and temporary dipole moment thereof must occur. In short, Raman activity of a vibration form is much different than its infrared activity.

Raman spectrums of the HAp and B-HAp samples are given in the FIGS. 6 and 7 respectively. All vibration peaks of PO₄³⁻ groups were also seen for both examples. PO₄³⁻ symmetric stretching peak was seen approximately at 961-962 cm⁻¹ are also characteristic for hydroxyapatites. Whereas peaks at 447, 433 and 603 cm⁻¹ indicate PO₄³⁻ bending vibrations in the structure. Asymmetric bending vibrations of PO₄³⁻ were found out approximately 1076, 1054, 1046 and 1030 cm⁻¹.

In the graphics given on the top left corner in the FIGS. 6 and 7, streching vibrations bend of the characteristic OH⁻ group in the structures were seen approximately at 3570 cm⁻¹.

BO₃³⁻ symmetric stretching vibration in the structure of B-Hap was seen at 912 cm⁻¹. Existence of boron in the structure is proved by Raman spectroscopy results once again.

The production capacities of microwave-assisted method was investigated to use HAp or B-HAp asbone filling material or coating. Production efficiencies were compared and the data were presented in the Table 7. As a result of the measurements, it was determined that maximum production quantity within the shortest time is obtained by microwave method and the highest amount was provided at 600 W power. In addition, efficiency was increased 3 times in comparison to the precipitation method at room temperature.

TABLE 7
Product yields of the HAp and B-Hap examples
which are produced by different methods

| | | | Product yield |
|---|---|---|---|
| Example | Condition | | (g/100 mL) |
| HAp | Precipitation at room | Raman shift, | 09 |
| | Conventional method | cm⁻¹ | 0.0293 ± 0.001 |
| | 90 W | | 0.0317 ± 0.005 |
| | 360 W | | 0.0324 ± 0.001 |
| | 600 W | | 0.0328 ± 0.002 |
| | 1200 W | | 0.0309 ± 0.002 |
| B-HAp | 600 W | | 0.0258 ± 0.002 |
| | Precipitation at | | No precipitation |
| | room temperature | | observed |

When hydroxyapatites were compared, it was determined that HAp produced under microwave irradiation was obtained more efficient than the hydroxyapatite produced by precipitation at room temperature for approximately three times. Also, no precipitation was observed at room temperature from SBF after boron addition. Production of B-HAp can only be realized in the existence of microwave or thermal applications. When both production yield and other characterization results were examined, it was seemed suitable to produce B-HAp by 600 W microwave. When HAp and B-HApproduced with the support of microwave were compared, production yield of B-HAp decreased due to change in SBF ion balance because of the borate ions included into the structure however it was seen that this amount is also sufficient for the target set when coatings are made.

Different analysis are made in order to characterize of the inventive B-HAp/chitosan and HAp/chitosan tissue scaffolds and the analysis results are given by figures and tables in the following.

For the purpose of showing existence of the coating made by biomimetic method, ATR-FTIR analysis of chitosan, HAp/chitosan, B-HAp/chitosan tissue scaffolds were made.

ATR-FTIR spectra attributed to the chitosan, HAp/chitosan, B-HAp/chitosan tissue scaffolds were given in the FIG. 8. The N-H band seen at approximately at 3459 cm⁻¹ in the spectrum of the pure chitosan given in the FIG. 8(a) belongs to symmetrical streching vibration. All characteristic peaks of C—O—C streching vibrations were seen at 1635 cm⁻¹ amid I, 1549 cm⁻ amid II, 1404 cm⁻¹ C—H band at 1151, 1065 and 1021 cm⁻¹. Wide peaks seen approximately at 3300-3500 cm⁻¹ in the composite tissue scaffolds spectrums in the FIG. 8b and c result from bound water included in the apatite structure and the moisture comprised by the example. Because the N—H symmetric stretching vibration attributed to chitosan is also located in this region, this band became more stronger in these scaffolds.

The band seen approximately at 1050 cm⁻¹ in the spectrums attributed to HAp/chitosan and B-HAp/chitosan became more stronger due to the fact that the peaks attributed to free amino groups in chitosan and phosphate groups in hydroxyapatite collide, and it expresses interaction of PO₄³⁻ and chitosan in the apatite structure. The peak about at 1490 cm⁻¹ in the spectrum belongs to the CO₃⁻² ion included into the apatite structure.

Peaks of chitosan were given in the Table 8. 955 cm⁻¹ P-O streching vibration, 635 cm⁻¹ O—H bending vibration belonging to hydroxyapatite were observed in composite structure in addition to these.

TABLE 8
Characteristic FTIR bands of chitosan.

| Peak Values (cm⁻¹) | Description |
|---|---|
| 3459-3454 | N—H symmetric stress |
| 1650-1642-1654-1632 | Amid I |
| 1595-1586 | Amid II |
| 1420 | C—H curvature |
| 1153, 1070, 1031 | C—O—C symmetric and asymmetric stress |
| 899 | C—O |

XRD analysis showed existence of the mineral phase which is created on the chitosan structures, with the coating works carried out within the scope of the present invention, XRD graphics of the chitosan, HAp/chitosan and B-HAp/chitosan are given in the FIG. 9.

Hydroxyapatite in composite structures was obtained by making coating 4 times at 9×30 s with 600 W microwave power. The characteristic peak of chitosan in all tissue scaffolds was seen about 2θ=10° ve2θ=20°. Whereas in HAp/chitosan and B-HAp/chitosan tissue scaffolds; 2θ=26° and 2θ=32° peaks, which are characteristic HAp peaks, were also determined as well as the characteristic chitosan peak. Wide HAp peaks indicate that the apatite in the composite structures obtained has a low crystallinity in other words the coating has a bone-like apatite structure.

When XRD data were examined, it was seen that coating made for both composite tissue scaffolds were sufficient.

Surface and sectional morphology of chitosan. HAp/chitosan, B-HAp/chitosan tissue scaffolds included within the scope of the present invention were evaluated by the SEM images. In the FIG. 10, the chitosan tissue scaffold obtained by freeze drying method has macro porous structure having interconnections.

No sufficient phase was composed in 1 time coating realized with the help of microwave inside the 10×SBF and boron-added 10×SBF (B-SBF) prepared (FIG. 11a). However, HAp nucleation regions necessary for realizing an efficient coating occurred (FIG. 11b). As a result of 4 coatings realized using fresh SBF in each coating, HAp was enabled to grow by stacking calcium and phosphate ions to the nucleation regions.

In FIG. 12, SEM images of HAp/chitosan and B-HAp/chitosan obtained by 4 coatings were given in different magnifications. It was observed that the surface was entirely coated by apatite structure and the coating was homogeneous in both tissue scaffold. In coatings made by SBF, formation of apatite was cauliflower-like morphology. When TEM images were examined, it was seen that apatite structures having a needle-like or a rod-like morphology usually take a nano-sphere form in coating on chitosan. Dimensions and length distribution of the hydroxyapatite formed on chitosan were determined by Image J programme using SEM images. When diameters of the clusters formed by the HAp crystals by combining were examined in 5.000× zoom, they were determined have diameters of 1.740±0.389 μm on average while diameters of the clusters formed by B-HAp crystals are 0.840±0.119 μm on average (when examined in 10,000× zoom) (FIG. 12 1). When the results were evaluated it was observed that boron addition causes diameter of hydroxyapatite crystal clusters to diminish and a more homogeneous and thin coating was obtained instead of big clusters in the coating

Due to three dimensional and porous structure of the tissue scaffolds, it was emphasized that, one of the most common problems about coating with SBF are lack of adequate coating and time consuming method. Providing mineralization in different bone substituting materials needed in treatment of various bone disorders is highly important. In order to overcome this problem, it was enabled to remove ait inside the pores under vacuum by immersing the tissue scaffolds prepared into SBF or B-SBF before coating. Thus it was ensured that inner pores are also filled with solution entirely and all surfaces are coated by means of the microwave irradiation while the pores are interacting with SBF solution. When the lateral sections belonging to the composite scaffolds prepared in the dimensions of 2mm×9mm for cell culture were examined it was seen that coating depth is sufficient in both HAp/chitosan (FIG. 13 a, b and c) and B-HAp/chitosan (FIG. 13d, e and f) tissue scaffolds and the pores are coated entirely however the coating on the upper surfaces have more.

Porosity of the tissue scaffolds were calculated and the results were given in the Table 9.

TABLE 9
Porosity values of the tissue scaffolds by percentage

| Tissue scaffolds | Porosity (%) |
|---|---|
| Chitosan | 89.5 ± 1.03 |
| HAp/chitosan | 81.9 ± 0.76 |
| B-HAp/chitosan | 85.7 ± 2.57 |

When the porosity was examined, it was seen that chitosan scaffold has highest porosity. Whereas porosity of HAp/chitosan and B-HAp/chitosan scaffolds are less than the chitosan. However, it was seen that porosity of B-HAp/chitosan scaffold was more when the composite scaffolds were compared between each other. It was concluded that this difference was because of the apatite in the structure of the B-HAp/chitosan coating consists of smalles crystals. Thus, pores were coated less and porosity was affected less. Due to magnitude of area to be deformed in scaffolds having high pore volume and dimension, some decrease of porosity in composite structures lead to increase of mechanical strength.

When all scaffolds were evaluated it was determined that porosity of 80% and more has a characteristic such that it ensures cell penetration into scaffold and also mass transfer for nutrient and metabolite. Materials that have highly porous structures are suitable in term of access to nutrients included in the medium for tissue engineering.

Chitosan, HAp/chitosan and B-HAp/chitosan included within the scope of the invention were evaluated in cell culture studies under static conditions for 21 days with preosteoblastic MC3T3-E1 cell line.

By the analysis carried out during culture; viability, morphologies and osteoblastic differentiations of preosteoblastic cells on tissue scaffolds were evaluated and effect of B-HAp scaffolds on differentiation were compared with other scaffolds.

In order to evaluate whether boron has any toxic effect on cells or not, cytotoxicity studies were done by L929 mouse fibroblast cell line.

In terms of evaluating whether boron amount included into B-HAp has any toxic effect or not, first of all cytotoxicity studies were done in accordance with ISO10993/EN 30993 standards by L929 fibroblast cell line supplied from HÜKÜK cell bank. After carrying out cell culture after 24 hours later (5×10⁴ cell/mL), stock boric acid solution was added into the culture medium in given concentrations such that it will comprise boron and the cell proliferation was followed for 3 days by MTT assay (FIG. 14).

The group wherein no boron (0 μg) is added into the culture medium was determined as control group. First-day absorbance values of the control group were accepted as %100 and a graphic of percentage viability against time was obtained by proportioning the values of the other group to the control group. When the first-day data were examined, no difference significant in terms of cell viability was observed among the groups. On the second-day, cell viability showed increase for all groups. However, cell viability in the group wherein 300 μg/mL boron is added is significantly less (p<0.01) when it is compared with the control group. On the 3^{rd} day of the MTT test, cell viability decreased significantly in the group wherein 300 μg/mL boron is added and the cell number continues rising in the other groups, similar to the control group. By the gravimetric analysis carried out after the coating works, it was measured that the boron resulting from the coating in the B-HAp/chitosan tissue scaffolds were approximately 20 μg.

In accordance with the data obtained by the cytotoxicity studies done, this value was defined as non-toxic and the B-HAp/chitosan tissue scaffolds produced was seen to be suitable for cell culture studies.

Within the scope of the cytotoxicity studies, crystal violet staining were performed for monitoring cell morphology besides cell viability (FIG. 15). As a result of the observations carried out under light microscope, comparative pictures of the groups wherein different boron concentrations are used and the control group were obtained. In the observations carried out for 3 days, it was determined that cells adhered to the culture plate surface healthily and spread. In line with the MTT results, a decrease was observed in the 3^{rd} day cell density in the group wherein 300 μg/mL boron concentration is added whereas it was determined that the cell density increased with each passing day in the other groups. In addition, it was detected that the cells exhibited fibroblastic morphology in all groups and maintained their nucleus integrity. Boron value of approximately 20 μg/scaffold was determined not to have any negative effect on cell proliferation and cell morphology as well.

Cell proliferation on chitosan, HAp/chitosan and B-HAp/chitosan scaffolds were examined and using mitochondrial activity assay test (MTT test) in a 21-day incubation period. The results were given in the FIG. 16 as optical density value.

When the MTT assay performed to determine cell viability in the studies done by tissue scaffolds, it was seen that all scaffolds were suitable for cell adhesion and proliferation. On the first 3 days of the culture, no significant difference was observed in terms of cell viability (p>0.05). On the 5^{th} day of the culture, the highest cell viability was reached in the -HAp/chitosan group (p<0.05). When the 7^{th} day MTT values were examined it was determined that cell viability in HAp/chitosan (p<0.05) and B-HAp/chitosan (p<0.01) tissue scaffolds are significantly compared to chitosan and the highest cell viability was in the B-HAp/chitosan group. No significant difference was found out between the groups on the 14^{th} and 21^{st} days. When the MTT were examined, it was seen that cell proliferation in both three groups increased logarithmically and it reached the highest level on the 14^{th} day.

Cell-material interactions and whether they are materials compatible in terms of biocompatibility or not were evaluated by the SEM images belonging to the chitosan, HAp/chitosan and B-HAp/chitosan tissue scaffold.

Morphologies of the MC3T3-E1 cells, which are cultured on chitosan on 4^{th} and 7^{th} and 14^{th} day of the culture are given in the FIG. 17. It was clearly seen in the FIG. 17. a, b and c that the cells adhere to the scaffold, spread and emigrate towards the pores with intrinsic connections. It is observed that cell proliferation increased on the 7^{th} day (FIG. 17 d and e) and the cells closed the scaffold pores almost entirely (FIG. 17 h and i) and also as seen in the FIG. 17 g and h. the cells established connection with one another by means of the cytoplasmic extensions thereof and exhibited a three dimensional proliferation.

4^{th}, 7^{th} and 14^{th} day images of the cells on HAp/chitosan are given in the FIG. 18 a-e, FIG. 18 f-i and FIG. 18 j-n, respectively. Interaction of the HAp coatings on the surface and the cells is clearly seen in the FIG. 18 d and e. The coatings with composite structure are seen between the cellular and extracellular matrices. And this is an indication of coating stability. In addition to the fact that the HAp coatings on the surface has no negative effect on cell adhesion or proliferation, Chen and et al. stated in their study that surface roughness increasing by nanotopographical details increased surface area and thus they created more adhesion area for cells.

Cells interacting with HAp both chemically and biologically head towards apatites by means of the cytoplasmic extensions. Thus, the cells turn onto differentiation by osteoinductive and osteoconductive effect of HAp. Mineral clusters and collagen fibres not seen on chitosan tissue scaffolds are prominently seen in HAp/chitosan scaffold on the 7^{th} and 14^{th} day (FIG. 18 h-i and FIG. 18 m, n).

SEM images (FIG. 18 j) were examined on day 14^{th}, it was observed that cells proliferating in accordance with the MTT data cover the scaffold surface entirely upon they proliferate too much. It was indicated in FIG. 18 n in a detailed way that the cells synthesized extracellular matrix intensely and generated nano-sized collagen fibres. In addition, it was observed that mineral accumulations on cell membranes grew more on the 14^{th} day (FIG. 18 l and m). By these results it was determined that HAp/chitosan scaffolds supported adhesion, proliferation, and osteoblastic differentiation of MC3T3-E1 cells.

SEM images of the B-HAp/chitosan on day 4^{th}, 7^{th} and 14^{th} were examined, it was shown by the FIGS. 19. a, f and j that intensity increased on the tissue scaffold. The cells were seen to adhere and spread on B-HAp coated surfaces and they can establish intercellular communication as of day 4^{th} on this morphology (FIG. 19 d). When MTT analysis results and SEM images were evaluated, it was seen that the boron included into the hydroxyapatite structure by biomimetic method did not lead to any toxic effect and it even supported cell adhesion and proliferation better than other groups. It is considered that the reason of this effect may be due to the fact that the boron or boron ions included into the structure of the apatite and also the real effect may depend on osteoinductive effect resulting from emission of boron ions occurring from the bone-like apatite.

Within the scope of the invention, gene expressions of collagen-I (KOL-I), RunX2, osteocalcin (OCN) and osteopontin (OPN) were determined by RT-PCR in order to determine osteogenic differences of the cells cultured on the tissue scaffolds.

During formation of a new bone, firstly cell proliferation occurs and the osteoblasts proliferating secrete KOL-I. Expression of type I collagen decreases in differentiation and maturation period.

When collagen-I expressions of cells on scaffolds were examined (FIG. 20), it was seen that KOL-I level in the chitosan scaffold did not exhibit any increase depending on the day. Whereas in HAp/chitosan and B-HAp/chitosan groups, KOL-I expression increased significantly from day 7^{th} until 14^{th} and reached the highest value (p<0.001). Also day values were examined on day 14^{th} in themselves it was seen that, KOL-I expression of HAp/chitosan group was more approximately 3 times and collagen amount of the B-HAp/chitosan group was more approximately 4-5 times when compared to chitosan (p<0.001). On day 21^{th}, KOL-I expression decreases in all groups and level of expression in B-HAp/chitosan group was more than other groups statistically (p<0.05).21. On day 21^{st}, it is clearly understood from the decreasing KOL-I levels that cells in the HAp and B-HAp chitosan scaffolds head towards differentiation. Yin and et al. stated in their study realized about effect of boron on osteogenic differentiation of human bone marrow stroma cells that addition of 1-1000 ng/mL boric acid into their medium increased KOL-I gene expression, which is an early differentiation marker of cells, compared to the controls groups on the 7^{th} day significantly.

In the study, the RunX2 gene expression initiating expressions of bone matrix proteins at early periods of osteoblast differentiation was examined (FIG. 21) and it was determined that expression level in HAp/chitosan and B-HAp/chitosan groups on the 7^{th} day was significantly higher compared to chitosan (p<0.001). RunX2 expression reached the highest level on the 14^{th} day for all groups. When RunX2 expressions were examined on day 14^{th} in themselves it was determined that the expression levels were 1.7 times more on HAp/chitosan group and 1.5 times more on B-HAp/chitosan compared to chitosan.

It was observed that bone tissue did not express in mice having RunX2 gene mutation which is a basic factor for osteoblast differentiation. This gene, which can also be active in mature osteoblasts, also affect osteocalcin, osteopontin and collagen-I expression levels.

0.1-1000 ng/mL, concentrations of boron are known to increase RunX2 gene expression of MC3T3-E1 cells. Wu and et al. indicated that osteoblasts on porous bioactive glass tissue scaffolds comprising boron increased RunX2 gene expressions of the culture on day 7^{th} and 14^{th}. This effect was predicted to result from boron ion released from the tissue scaffold.

Osteocalcin, which is a marker of formation of mature osteoblast, occurs in the late periods of differentiation. The status in vitro medium is as it exists in in vivo medium, early differentiation markers increase when the cell proliferation decreases and osteocalcin is expressed in the last stage.

When osteocalcin expressions were examined (FIG. 22) it was seen that expression level in all groups increased logarithmically from day 7^{th} to 21^{st} and reaches the highest level thereof on day 21^{st}. On day 14^{th}, osteocalcin level in B-HAp/chitosan is significantly higher than the other groups (p<0.05). On day osteocalcin level on HAp/chitosan was 1.6 times of chitosan whereas osteocalcine level in B-HAp/chitosan was 1.9 times of chitosan. The highest osteocalcin level in B-HAp/chitosan scaffold as a marker that differentiation and mineralization are supported. These results were also supported by the mineralized strictures in the SEM images. Grausova and et al. stated in their culture study, which was carried out by MG 63 osteoblast-like cells on diamond films contributed by boron up to 6700 ppm that osteocalcin gene expressions of cells increase as boron contribution of films increase.

Osteopontin is an important protein acting in bone formation. It inhibits mineralization thereof in late periods of osteogenic differentiation and thus acts as a negative regulator.

When osteopontin expressions were examined (FIG. 23), it was seen that the highest expression level was seen in B-HAp/chitosan group on 7^{th} day. In all groups, osteopontin expression increased depending on the day. On day 14^{th} day. OPN expression on B-HAp/chitosan is approximately 2 times of chitosan and approximately 1.4 times of HAp/chitosan. On day 21^{st}, OPN expression was significantly higher when compared to chitosan on HAp/chitosan and B-HAp/chitosan.

Hakki and et al. examined gene expressions in MC3T3-E1 wherein they applied boron concentration of 0-1000 μg/mL and detected that 100 and 1000 ng/mL boron concentration increased osteopontin on 8^{th} day.

When RT-PCR results were evaluated in general, it was detected that HAp/chitosan and B-HAp/chitosan tissue scaffolds supported differentiation of MC3T3 bone precursor cells and extracellular matrix formation however this effect is more obvious due to boron in B-HAp/chitosan tissue scaffolds.

Within the framework of these basic concepts, it is possible to develop various embodiments of the inventive method for producing HAp (hydroxyapatite)/boron-doped HAp and developing composite tissue scaffolds. The invention cannot be limited to the examples described herein: it is essentially as defined in the claims.

1. A method characterized in that following the steps of:
preparing a SBF (simulated body fluid) solution which comprises a Ca/P (calcium/phosphate) concentration 10 times more than mood plasma;
adding NaHCO₃ into the solution before the precipitation process;
to exposure the solution to a microwave process and carrying out the precipitation process
in order to obtain HAp for bone tissue repair by biomimetic method.

2. A method according to claim 1, characterized in that there are components of NaCl, KCl, CaCl₂, 2H₂O, MgCl₂.6H₂O and NaH₂PO₄.H₂O in the solution.

3. A method according to claim 1, characterized in that firstly NaCl, secondly KCl, thirdly CaCl₂.2H₂O, then MgCl₂.6H₂O and lastly NaH₂PO₄. H₂O are added when preparing the solution.

4. A method according to claim 1, characterized in that first five salt components are dissolved in a magnetic stirrer within 800 mL distilled water respectively and it is completed to 1000 by addition of 200 mL distilled water in preparation of solution.

5. A method according to claim 4, characterized in that 100 mL stock SBF solution is taken into a glass beaker and added NaHCO₃ such that its concentration will be 10 mM before the precipitation process.

6. A method according to claim 1, characterized in that precipitation process occurs by applying the microwave processes to the solution by various powers, various times and number of repetition.

7. A method according to claim 1, characterized in that the solution is washed and allowed to dry in order to remove other undesirable phases after the precipitation process.

8. A method according to claim 7, characterized in that the solution is washed by being centrifuged at 5,000 rpm for 5 minutes within ethanol for 2 times and distilled water for 3 times and it is allowed to dry in drying-oven at 37° C. temperature for the purpose of removing other undesirable phases.

9. A method according to claim 1, characterized in that salts are added in order to prepare a 10×SBF-like solution after H₃BO₃ (boric acid) and NaOH (sodium hydroxide) are added into 1000 ultrapure water so as to obtain a bone-like B-HAp.

10. A method according to claim 9, characterized in that firstly H₃BO₃ (boric acid) is added into the 10×SBF solution in order to provide the bone-like HAp boron additive precipitated from the simulated body fluid.

11. A method according to claim 9, characterized in that addition of NaHCO₃ made into the solution for the purpose of providing a suitable pH so that the precipitation process occurs.

12. A method according to claim 11, characterized in that the solution is washed and allowed to dry in order to and then B-HAp is obtained in order to remove other undesirable phases after the precipitation process.

13. A method according to claim 11, characterized in that the coating process is carried out by subjecting the chitosan tissue scaffolds, which are prepared by freeze drying method, to microwave process in SBF solution and type composite scaffolds are obtained for producing HAp/chitosan and/or B-HAp/chitosan coated composite tissue scaffolds with the help of microwave.

14. A method according to claim 13, characterized in that firstly 2% (w/v) chitosan (75-85% degree of deacetylation) solution of chitosan tissue scaffolds are prepared using distilled water comprising 0.2 M acetic acid in the freeze drying process.

15. A method according to claim characterized in that the solution obtained is filtered to eliminate impurities and it is frozen at −20° C. after being poured into 24-well Petri dishes.

16. A method according to claim 15, characterized in that the chitosan tissue scaffolds are subjected to the process of “freeze drying” at −80° C. using lyophilizer (freeze dryer).

17. A method according to claim 16, characterized in that the scaffolds are hold in 96% (v/v) ethanol solution for 1 day whereas in 70% (v/v) ethanol solution for 1 hour for stabilization of the chitosan tissue scaffolds obtained.

8. A method according to claim 13, characterized in that after they are cut by dies in 2 mm×9 mm sizes the tissue scaffolds are precipitated in a vacuum drying-oven within stock solutions wherein no NaHCO₃ is added, it is ensured that air inside thereof is taken out and all pores are contacted with SBF solutions.

19. A method according to claim 18, characterized in that the scaffolds are transferred into 100 mL SBF and B-SBF solutions wherein NaHCO₃ is added, coating is carried out with the help of microwave such that there will be 7 scaffolds/100 mL solution (SBF and B-SBF).
